(19) United States
(12) Patent Application Publication
(10) Pub. No.: US 2022/0249856 A1
(43) Pub. Date: Aug. 11, 2022

(54) METHOD OF TREATING TUMORS AND CAUSING FULL REGROWTH AND REGENERATION OF TISSUE

(71) Applicant: Dan Dietz, Richmond, TX (US)

(72) Inventor: Dan Dietz, Richmond, TX (US)

(21) Appl. No.: 17/173,945

(22) Filed: Feb. 11, 2021

(51) Int. Cl. A61N 2/00; A61K 41/00

(52) U.S. Cl. CPC A61N 2/004; A61K 41/0028

(57) ABSTRACT

The method of treating tumors and causing full regrowth and regeneration of tissue provides for the treatment and removal of tumors through application of a static magnetic field to flesh containing a tumor, as well as quickly and effectively healing, regenerating and regrowing the tissue following treatment of the tumor. At least two magnets are positioned within a treatment region adjacent to the tumor. Flesh containing a tumor is clamped between a first annular magnet and a second annular magnet, such that the flesh is sandwiched therebetween, with each of the first and second annular magnets surrounding the tumor. Treatment of the tumor causes the tumor to become necrotic and separate from the flesh. The application of the static magnetic field is further found to effect enhanced healing, regeneration and regrowth of the tumor-free tissue.

### BACKGROUND

### 1. Field

The disclosure of the present patent application relates to cancer and tumor treatment, and particularly to a method of treating tumors and causing full regrowth and regeneration of tissue through the application of a static magnetic field, as well as providing for healing, regrowth and regeneration of the tissue following the tumor treatment. As a non-limiting example, the static magnetic field may be applied by clamping tumor-containing flesh between a pair of annular magnets. Application of a static magnetic field to the tumor in this manner causes the tumor to become necrotic and separate from the flesh. The application of the static magnetic field is found to further effect enhanced healing, regrowth and regeneration of the tumor-free tissue.

### 2. Description of the Related Art

Malignant tumors are presently treated by a variety of different methods, including, but not limited to, chemotherapy, radiation therapy, surgery, immunotherapy and laser therapy. Although proven to be effective, each of these methods is typically seen to be undesirable for the patient due to the inherent risks of the treatment itself as well as the various negative side effects associated with each treatment. Each of these methods is also costly, time consuming and requires highly specialized equipment and medical practitioners.

The use of non-ionizing magnetic fields to treat tumors has shown promise in a number of in vitro and animal studies. Such treatment is not painful, relies only on conventional magnets or electromagnets and, unlike chemotherapy and ionizing radiation treatments, does not also harm normal tissues. Similarly, blocking tumor blood vessel growth, and the associated starving of tumors of their blood supply, is of great interest for tumor treatment due to the lack of damage to healthy tissue and general absence of pain and side effects for the patient. It would obviously be desirable to be able to effectively treat tumors using non-invasive and easily administered magnetic and restricted blood flow techniques. Thus, a method of treating tumors and causing full regrowth and regeneration of tissue solving the aforementioned problems is desired.

### SUMMARY

The method of treating tumors and causing full regrowth and regeneration of tissue provides for the treatment and removal of tumors through application of a static magnetic field to flesh containing a tumor, as well as quickly and effectively healing, regrowing and regenerating the tissue following treatment of the tumor. At least two magnets are positioned within a treatment region adjacent to the tumor. It should be understood that the term “tumor”, as used herein, refers to any type of neoplasm of tissue, including benign neoplasms, in situ neoplasms, malignant neoplasms, and neoplasms of uncertain or unknown behavior. In one embodiment of the method of treating tumors and causing full regrowth and regeneration of tissue, flesh containing a tumor is clamped between a first annular magnet and a second annular magnet through mutual attraction of the first and second annular magnets. The tumor is positioned within a region defined by central openings respectively formed through the first and second annular magnets, with the flesh being sandwiched between the first and second magnets, and with each of the first and second annular magnets surrounding the tumor. The treatment with the pair of annular magnets is continued until the tumor becomes necrotic and separates, or may be separated from, the remaining healthy tissue. As will be described in greater detail below, the application of the static magnetic field is found to further effect enhanced healing, regrowth and regeneration of the tumor-free tissue.

Experiments have shown that following treatment with the first and second annular magnets, as described above, the tissue at the site of treatment is quick to heal, regenerate and regrow into healthy tumor-free tissue. The method of treating tumors and causing full regrowth and regeneration of tissue may hold certain types of immune-related cells within the treatment area. The method of treating tumors and causing full regrowth and regeneration of tissue may also hold the body's stem cells within the treatment area for an extended period of time, effecting the enhanced healing, regrowth and regeneration of the tumor-free tissue.

With regard to application of the static magnetic field, static magnetic fields applied to skin have been shown to decrease the rate of skin blood perfusion. The static magnetic fields generated by the first and second annular magnets are expected to slow blood flow in the treatment region by between approximately 20% and approximately 40%. Thus, in addition to depriving the tumor of oxygen, the combination of slowing of blood flow, decrease in the rate of skin blood perfusion, and at least partial entrainment of the blood within the treatment region may hold immune effector cells in the treatment region for an extended period of time. Similar to conventional immunotherapy, the immune effector cells which may be held within the treatment region include the body's own lymphocytes, macrophages, dendritic cells, natural killer cells, cytotoxic T lymphocytes, etc., which work together to defend the body against cancer by targeting abnormal antigens expressed on the surface of tumor cells.

In order to take advantage of the above effects, it should be understood that the static magnetic fields may also be applied in an alternative manner. As a non-limiting example, two or more magnets may be secured to a band, strap, harness or the like to be held against the body, such that at least two of the magnets are held at a sufficient angle at the treatment site to slow blood flow, decrease the rate of skin blood perfusion, and at least partially entrain the blood within the treatment region.

These and other features of the present subject matter will become readily apparent upon further review of the following specification.

### BRIEF DESCRIPTION OF THE DRAWINGS

FIG. 1 illustrates a pair of annular magnets being applied to flesh containing a tumor, as an initial step of the method of treating tumors and causing full regrowth and regeneration of tissue.

FIG. 2 is side view in section of the pair of annular magnets clamping the flesh containing the tumor.

FIG. 3 is a top view illustrating a top one of the pair of annular magnets surrounding the tumor contained in the flesh.

Similar reference characters denote corresponding features consistently throughout the attached drawings.

### DETAILED DESCRIPTION OF THE PREFERRED EMBODIMENTS

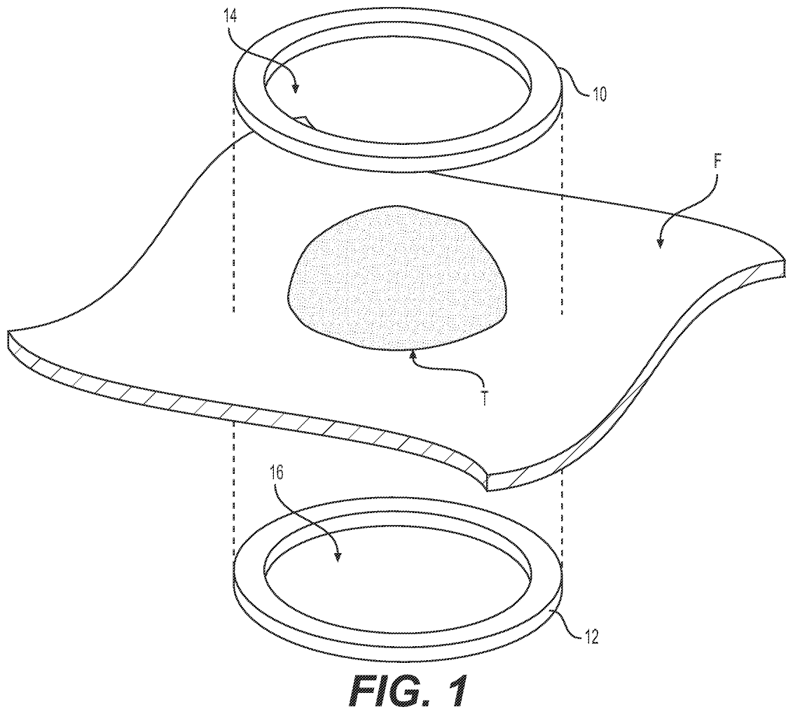

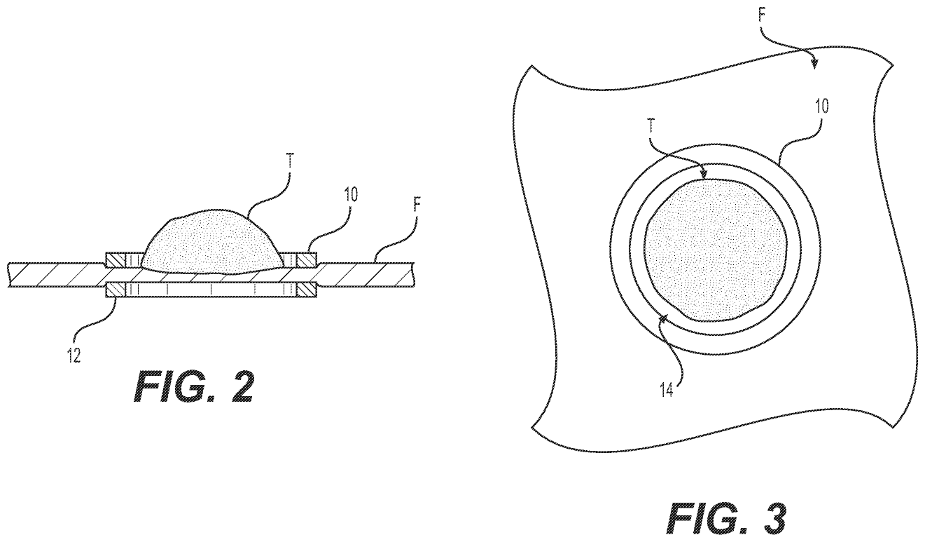

The method of treating tumors and causing full regrowth and regeneration of tissue provides for the treatment and removal of tumors T through application of a pair of annular magnets 10, 12. It should be understood that the term “tumor”, as used herein, refers to any type of neoplasm of tissue, including benign neoplasms, in situ neoplasms, malignant neoplasms, and neoplasms of uncertain or unknown behavior. FIGS. 1-3 illustrate an exemplary portion of animal flesh F which contains a tumor T. It should be understood that the section and type of flesh shown in FIGS. 1-3 is shown for exemplary purposes only. Similarly, it should be understood that the size, shape and type of tumor shown in FIGS. 1-3 is shown for exemplary purposes only. As shown in FIGS. 1 and 2, flesh F is clamped between a first annular magnet 10 and a second annular magnet 12 through mutual attraction of the first and second annular magnets 10, 12. As shown, the tumor T is positioned within a region defined by central openings 14, 16, which are respectively formed through the first and second annular magnets 10, 12. The flesh F is sandwiched between the first and second magnets 10, 12, with each of the first and second annular magnets 10, 12 surrounding the tumor T.

By clamping the first and second annular magnets 10, 12 around tumor T, tumor T is exposed to a static non-ionizing magnetic field, and the clamping of flesh F between the first and second annular magnets 10, 12 further restricts blood flow to tumor T. The opposite poles of the first and second annular magnets 10, 12 are oriented toward one another to produce an attracting force between first and second annular magnets 10, 12, thus encapsulating tumor T within, and between, the pair of annular magnets 10, 12. Blood flow to tumor T is restricted by the pinching of flesh F by the attraction between first and second annular magnets 10, 12. The blood flow to tumor T may be further restricted due to application of the static magnetic field generated by the first and second annular magnets 10, 12. Static magnetic fields applied to skin have been shown to decrease the rate of skin blood perfusion, as discussed, for example, in Mayrovitz, Harvey N., and Edye E. Groseclose. “Effects of a static magnetic field of either polarity on skin microcirculation.” Microvascular research 69.1-2 (2005): 24-27.

The static magnetic fields generated by the first and second annular magnets 10, 12 are expected to slow blood flow in the treatment region by between approximately 20% and approximately 40%. Thus, in addition to depriving the tumor T of oxygen, the combination of slowing of blood flow, decrease in the rate of skin blood perfusion, and at least partial entrainment of the blood within the treatment region may hold immune effector cells in the treatment region for an extended period of time. Similar to conventional immunotherapy, the immune effector cells which may be held within the treatment region include the body's own lymphocytes, macrophages, dendritic cells, natural killer cells, cytotoxic T lymphocytes, etc., which work together to defend the body against cancer by targeting abnormal antigens expressed on the surface of tumor cells.

In order to measure the strength of the magnetic field generated by the first and second annular magnets 10, 12, a sealed gaussmeter, magnetometer or any other suitable type of magnetic sensor may be implanted or embedded in flesh F at, or adjacent to, the treatment site. It should be understood that the first and second annular magnets 10, 12 may be any suitable type of magnets. It should be further understood that one of the first and second annular magnets 10, 12 may be replaced by an annular member formed from a paramagnetic material. It should be further understood that the size and relative dimensions of the first and second annular magnets 10, 12 are shown in FIGS. 1-3 for exemplary and illustrative purposes only.

In an experiment, a pair of annular magnets was used to treat a tumor located in the lip of a dog. Each annular magnet was a neodymium magnet, with a thickness of 0.095 inches, an inner diameter of 1.296 inches, and an outer diameter of 1.652 inches. At the surface (i.e., at the point of contact with the magnetometer), each annular magnet generated a magnetic field of 1.35 kG. The dog was approximately 10 years old, with a tumor which had grown over the course of approximately 10 months. After 19 continuous hours of treatment with the pair of annular magnets, as described above, the tumor became necrotic and treatment with the pair of magnets was ceased. Five days after cessation of the treatment, the necrotic tumor tissue began to separate from the healthy lip tissue and flake off. The tissue at the site of treatment was quick to heal, regenerate and regrow into healthy tumor-free tissue, with regrowth and regeneration of healthy tissue being clearly evident one month after cessation of the treatment with the pair of annular magnets. During treatment, the dog did not appear to show any signs of pain, distress, blood loss or additional generation of any bodily fluids. As described above, the method of treating tumors and causing full regrowth and regeneration of tissue may hold certain types of immune-related cells within the treatment area. The method of treating tumors and causing full regrowth and regeneration of tissue may also hold the body's stem cells within the treatment area for an extended period of time, effecting the observed enhanced healing, regrowth and regeneration of the tumor-free tissue.

Additionally, it should be understood that the magnetic treatment of tumors and cancers may be performed at any suitable site on the body, or at multiple sites, including full body immersion in a magnetic field. Further, it should be understood that the magnets applied to the flesh do not need to be free magnets; i.e., they could be integrated into wearable belts, garments, attached to tools and frames, integrated into jewelry and the like. Thus, in order to take advantage of the above-described magnetic field-based effects, it should be understood that the magnetic fields may be applied in an alternative manner. As a non-limiting example, two or more magnets may be secured to a band, strap, harness or the like to be held against the body, such that at least two of the magnets are held at a sufficient angle at the treatment site to slow blood flow, decrease the rate of skin blood perfusion, and at least partially entrain the blood within the treatment region.

It is to be understood that the method of treating tumors and causing full regrowth and regeneration of tissue is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

What is claimed is:

1. A method of treating a tumor and causing full regrowth and regeneration of tissue, comprising:
clamping flesh containing the tumor between a first annular static magnet providing a first static magnetic field and a second annular static magnet providing a second static magnetic field, wherein the tumor is positioned within a region defined by central openings respectively formed through the first annular static magnet and the second annular static magnet.

2. The method as recited in claim 1, wherein the step of clamping the flesh containing the tumor comprises sandwiching the flesh between the first annular static magnet and the second annular static magnet.

3. The method as recited in claim 2, wherein the step of clamping the flesh containing the tumor further comprises positioning the first annular static magnet and the second annular static magnet to surround the tumor.

4. The method as recited in claim 1, wherein the tumor is a malignant tumor.

5. The method as recited in claim 1, wherein the tumor is a benign tumor.

6. A method of treating a tumor and causing regrowth and regeneration of tissue, comprising:
applying a static magnetic field to flesh containing said tumor by positioning at least two magnets within a treatment region adjacent to said tumor, wherein the least two magnets are clamping the flesh to provide said static magnetic field on said tumor, and said static magnetic field is from mutual attraction of the at least two magnets.

7. The method as recited in claim 1, wherein said first static magnetic field and said second static magnetic field overlaps on said tumor.

8. The method as recited in claim 6, wherein said at least two magnets are positioned to face each other.
